(19) United States
(12) Patent Application Publication
(10) Pub. No.: US 2022/0022798 A1
(43) Pub. Date: Jan. 27, 2022

(54) Waveform Analysis And Detection Using Machine Learning Transformer Models

(71) Applicants: Nant Holdings IP, LLC, Culver City, CA (US); NantCell, Inc., Culver City, CA (US)

(72) Inventors: Patrick SOON-SHIONG, Los Angeles, CA (US); Bing SONG, La Canada, CA (US); Nicholas James WITCHEY, Laguna Hills, CA (US)

(21) Appl. No.: 17/376,955

(22) Filed: Jul. 15, 2021

Related U.S. Application Data
(60) Provisional application No. 63/055,686, filed on Jul. 23, 2020.

(51) Int. Cl. A61B 5/352; A61B 5/00; G06N 3/08

(52) U.S. Cl. CPC A61B 5/352; G06N 20/00; G06N 3/088; A61B 5/7232

(57) ABSTRACT

A computerized method of analyzing a waveform using a machine learning transformer model includes obtaining labeled waveform training data and unlabeled waveform training data, supplying the unlabeled waveform training data to the transformer model to pre-train the transformer model by masking a portion of an input to the transformer model, and supplying the labeled waveform training data to the transformer model without masking a portion of the input to the transformer model to fine-tune the transformer model. Each waveform in the labeled waveform training data includes at least one label identifying a feature of the waveform. The method also includes supplying a target waveform to the transformer model to classify at least one feature of the target waveform. The at least one classified feature corresponds to the least one label of the labeled waveform training data.

### CROSS-REFERENCE TO RELATED APPLICATION

This application claims the benefit of U.S. Provisional Application No. 63/055,686, filed on Jul. 23, 2020. The entire disclosure of the above application is incorporated herein by reference.

### FIELD

The present disclosure relates to waveform analysis and detection using machine learning transformer models, and particularly to analysis and detection of electrocardiogram waveforms.

### BACKGROUND

With low-cost biosensor devices available, such as electrocardiogram (ECG or EKG) devices, electroencephalogram (EEG) devices, etc., more and more patient recordings are taken every year. For example, more than 300 million ECGs are recorded annually. Each ECG typically involves multiple electrodes positioned at different locations on a patient, in order to measure signals related to heart activity. The electrode measurements create an ECG waveform that may be analyzed by medical professionals.

Separately, a Bidirectional Encoder Representations from Transformers (BERT) model is a self-supervised machine learning model that was developed for natural language processing. The BERT model includes one or more encoders for processing input data and providing a classified output.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

A computerized method of analyzing a waveform using a machine learning transformer model includes obtaining labeled waveform training data and unlabeled waveform training data, supplying the unlabeled waveform training data to the transformer model to pre-train the transformer model by masking a portion of an input to the transformer model, and supplying the labeled waveform training data to the transformer model without masking a portion of the input to the transformer model to fine-tune the transformer model. Each waveform in the labeled waveform training data includes at least one label identifying a feature of the waveform. The method also includes supplying a target waveform to the transformer model to classify at least one feature of the target waveform. The at least one classified feature corresponds to the least one label of the labeled waveform training data.

In other features, the method includes obtaining categorical risk factor data, obtaining numerical risk factor data, embedding categorical risk factor data and concatenating the embedded categorical risk factor data with the numerical risk factor data to form a concatenated feature vector. The method may include supplying the concatenated feature vector to the transformer model to increase an accuracy of the at least one classified feature.

In other features, the unlabeled waveform training data, the labeled waveform training data, and the target waveform each comprise an electrocardiogram (ECG) waveform recorded from a patient, the categorical risk factor data includes a sex of the patient, and the numerical risk factor data includes at least one of an age of the patient, a height of the patient, and a weight of the patient. In other features, the categorical risk factor data includes multiple groups of categorical values, each group is encoded using one-hot encoding, and embedding the categorical risk factor data includes combining each of the encoded groups into a combined encoded vector and then feeding the combined encoded vector to a neural network to output an embedded categorical risk factor vector.

In other features, the unlabeled waveform training data, the labeled waveform training data, and the target waveform each comprise an electrocardiogram (ECG) waveform recorded from a patient, the at least one label of each waveform in the labeled waveform training data includes at least one of a detected heart arrhythmia, a P wave and a T wave, and the at least one classified feature includes the at least one of a detected heart arrhythmia, a P wave and a T wave.

In other features, the transformer model comprises a Bidirectional Encoder Representations from Transformers (BERT) model. In other features, supplying the unlabeled waveform training data to pre-train the transformer model and supplying the labeled waveform training data to fine-tune the transformer model each include periodically relaxing a learning rate of the transformer model by reducing the learning rate during a specified number of epochs and then resetting the learning rate to an original value before running a next specified number of epochs.

In other features, the unlabeled waveform training data includes daily seismograph waveforms, the labeled waveform training data includes detected earthquake event seismograph waveforms, and the at least one classified feature includes a detected earthquake event. In other features, the labeled waveform training data, the unlabeled waveform training data, and the target waveform each include at least one of an automobile traffic pattern waveform, a human traffic pattern waveform, an electroencephalogram (EEG) waveform, a network data flow waveform, a solar activity waveform, and a weather waveform. In other features, the transformer model is located on a processing server, the target waveform is stored on a local device separate from the processing server, and the method further includes compressing the target waveform and transmitting the target waveform to the processing server for input to the transformer model.

In other features, a computer system includes memory configured to store unlabeled waveform training data, labeled waveform training data, a target waveform, a transformer model, and computer-executable instructions, and at least one processor configured to execute the instructions. The instructions include obtaining labeled waveform training data and unlabeled waveform training data, supplying the unlabeled waveform training data to the transformer model to pre-train the transformer model by masking a portion of an input to the transformer model, and supplying the labeled waveform training data to the transformer model without masking a portion of the input to the transformer model to fine-tune the transformer model. Each waveform in the labeled waveform training data includes at least one label identifying a feature of the waveform. The instructions also include supplying a target waveform to the transformer model to classify at least one feature of the target waveform. The at least one classified feature corresponds to the least one label of the labeled waveform training data.

In other features, the instructions include obtaining categorical risk factor data, obtaining numerical risk factor data, embedding categorical risk factor data and concatenating the embedded categorical risk factor data with the numerical risk factor data to form a concatenated feature vector, and supplying the concatenated feature vector to the transformer model to increase an accuracy of the at least one classified feature.

In other features, the unlabeled waveform training data, the labeled waveform training data, and the target waveform each comprise an electrocardiogram (ECG) waveform recorded from a patient, the categorical risk factor data includes a sex of the patient, and the numerical risk factor data includes at least one of an age of the patient, a height of the patient, and a weight of the patient. In other features, the categorical risk factor data includes multiple groups of categorical values, each group is encoded using one-hot encoding, and embedding the categorical risk factor data includes combining each of the encoded groups into a combined encoded vector and then feeding the combined encoded vector to a neural network to output an embedded categorical risk factor vector.

In other features, the unlabeled waveform training data, the labeled waveform training data, and the target waveform each comprise an electrocardiogram (ECG) waveform recorded from a patient, the at least one label of each waveform in the labeled waveform training data includes at least one of a detected heart arrhythmia, a P wave and a T wave, and the at least one classified feature includes the at least one of a detected heart arrhythmia, a P wave and a T wave.

In other features, the transformer model comprises a Bidirectional Encoder Representations from Transformers (BERT) model. In other features, supplying the unlabeled waveform training data to pre-train the transformer model and supplying the labeled waveform training data to fine-tune the transformer model each include periodically relaxing a learning rate of the transformer model by reducing the learning rate during a specified number of epochs and then resetting the learning rate to an original value before running a next specified number of epochs.

In other features, the unlabeled waveform training data includes daily seismograph waveforms, the labeled waveform training data includes detected earthquake event seismograph waveforms, and the at least one classified feature includes a detected earthquake event. In other features, the labeled waveform training data, the unlabeled waveform training data, and the target waveform each include at least one of an automobile traffic pattern waveform, a human traffic pattern waveform, an electroencephalogram (EEG) waveform, a network data flow waveform, a solar activity waveform, and a weather waveform. In other features, the transformer model is located on a processing server, the target waveform is stored on a local device separate from the processing server, and the instructions further include compressing the target waveform and transmitting the target waveform to the processing server for input to the transformer model.

In other features, a non-transitory computer-readable medium storing processor-executable instructions, and the instructions include obtaining labeled waveform training data and unlabeled waveform training data, supplying the unlabeled waveform training data to a transformer model to pre-train the transformer model by masking a portion of an input to the transformer model, and supplying the labeled waveform training data to the transformer model without masking a portion of the input to the transformer model to fine-tune the transformer model. Each waveform in the labeled waveform training data includes at least one label identifying a feature of the waveform. The instructions also include supplying a target waveform to the transformer model to classify at least one feature of the target waveform. The at least one classified feature corresponds to the least one label of the labeled waveform training data.

In other features, the instructions include obtaining categorical risk factor data obtaining numerical risk factor data, embedding categorical risk factor data and concatenating the embedded categorical risk factor data with the numerical risk factor data to form a concatenated feature vector, and supplying the concatenated feature vector to the transformer model to increase an accuracy of the at least one classified feature.

In other features, the unlabeled waveform training data, the labeled waveform training data, and the target waveform each comprise an electrocardiogram (ECG) waveform recorded from a patient, the categorical risk factor data includes a sex of the patient, and the numerical risk factor data includes at least one of an age of the patient, a height of the patient, and a weight of the patient. In other features, the categorical risk factor data includes multiple groups of categorical values, each group is encoded using one-hot encoding, and embedding the categorical risk factor data includes combining each of the encoded groups into a combined encoded vector and then feeding the combined encoded vector to a neural network to output an embedded categorical risk factor vector.

In other features, the unlabeled waveform training data, the labeled waveform training data, and the target waveform each comprise an electrocardiogram (ECG) waveform recorded from a patient, the at least one label of each waveform in the labeled waveform training data includes at least one of a detected heart arrhythmia, a P wave and a T wave, and the at least one classified feature includes the at least one of a detected heart arrhythmia, a P wave and a T wave.

In other features, the transformer model comprises a Bidirectional Encoder Representations from Transformers (BERT) model. In other features, supplying the unlabeled waveform training data to pre-train the transformer model and supplying the labeled waveform training data to fine-tune the transformer model each include periodically relaxing a learning rate of the transformer model by reducing the learning rate during a specified number of epochs and then resetting the learning rate to an original value before running a next specified number of epochs.

In other features, the unlabeled waveform training data includes daily seismograph waveforms, the labeled waveform training data includes detected earthquake event seismograph waveforms, and the at least one classified feature includes a detected earthquake event. In other features, the labeled waveform training data, the unlabeled waveform training data, and the target waveform each include at least one of an automobile traffic pattern waveform, a human traffic pattern waveform, an electroencephalogram (EEG) waveform, a network data flow waveform, a solar activity waveform, and a weather waveform. In other features, the transformer model is located on a processing server, the target waveform is stored on a local device separate from the processing server, and the instructions further include compressing the target waveform and transmitting the target waveform to the processing server for input to the transformer model.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a functional block diagram of an example system for waveform analysis using a machine learning transformer model.

FIG. 2 is a functional block diagram of pre-training an example transformer model for use in the system of FIG. 1.

FIG. 3 is a functional block diagram of fine-tuning training for the example transformer model of FIG. 2.

FIG. 4 is a flowchart depicting an example method of training a transformer model for waveform analysis.

FIG. 5 is a flowchart depicting an example method of using a transformer model to analyze an electrocardiogram (ECG) waveform.

FIG. 6 is an illustration of an example ECG waveform including P and T waves.

FIG. 7 is a functional block diagram of a computing device that may be used in the example system of FIG. 1.

In the drawings, reference numbers may be reused to identify similar and/or identical elements.

### DETAILED DESCRIPTION

### Introduction

With low-cost biosensor devices available, such as electrocardiogram (ECG or EKG) devices, electroencephalogram (EEG) devices, etc., more and more patient recordings are taken every year. For example, more than 300 million ECGs are recorded annually. ECG diagnostics may be improved significantly if a large amount of recorded ECGs are used in a self-learning data model, such as a transformer model. For example, the Bidirectional Encoder Representations from Transformers (BERT) model may be used where a large amount of unlabeled ECG data is used to pre-train the model, and a smaller portion of labeled data ECG data (e.g., with heart arrhythmia indications classified for certain waveforms, with P and T waves indicated on certain waveforms, etc.) is used to fine-tune the model. Further, additional health data is abundant from mobile applications such as daily activity, body measurement, risk factors, etc., which may be incorporated with the ECG waveform data to improve cardiogram diagnostics, waveform analysis, etc. Similarly, techniques disclosed herein may be applied to other types of sensor data that has a waveform structure, such as music, etc., and different types of data modalities may be converted to other waveform structures.

In various implementations, a transformer model (e.g., an encoder-decoder model, an encoder only model, etc.) is applied to a waveform such as an ECG, an electroencephalogram (EEG), other medical waveform measurements, etc. For example, when a vast amount of unlabeled waveforms are available, such as general ECGs, the large amount of data may be used to pre-train the transformer model to improve accuracy of the transformer model.

If available, additional health data may be integrated in the model, such as risk factors from an electronic health record (EHR), daily activity form a smart phone or watch, clinical outcomes, etc. While EHRs may include specific patient data, larger datasets may exist for cohorts. This additional health data may improve the diagnostic accuracy of the transformer model. For example, the transformer model may be used to identify conditions such as a heart arrhythmia, may use an algorithm such as Pan Thompkins to generate a sequence for detecting an R wave in the ECG waveform and then detect P and T waves, etc.

In various implementations, a large scale client-server architecture may be used for improved efficiency and communication between devices. For example, if a local device has enough memory and processing power, the transformer model may run on the local device to obtain desired diagnostics. Results may then be sent to a server. In situations where the local device does not have enough memory or processing power to run the transformer model in a desired manner, the local device may compress the waveform through FFT or other type of compression technique and send the compressed data with additional risk factors, daily activity, etc., to the server. This allows for a scalable solution by combining a local-based system and a client-server-based system. In some implementations, the FFT compressed waveform may be supplied directly to the BERT model without decompressing to obtain the original waveform. For example, discrete wavelet transform has been successfully applied for the compression of ECG signals, where correlation between the corresponding wavelet coefficients of signals of successive cardiac cycles is utilized by employing linear prediction. Other example techniques include the Fourier transform, time-frequency analysis, etc. Techniques described herein may be applied in larger ecosystems, such as a federated learning system where the models are built on local systems using local private data, and then aggregated in a central location while respecting privacy and HIPAA rules.

In various implementations, the transformer models may be applied to analyze waveforms for earthquake and shock detection, for automobile and human traffic pattern classification, for music or speech, for electroencephalogram (EEG) analysis such as manipulating artificial limbs and diagnosing depression and Alzheimer's disease, for network data flow analysis, for small frequency and long wavelength pattern analysis such as solar activities and weather patterns, etc.

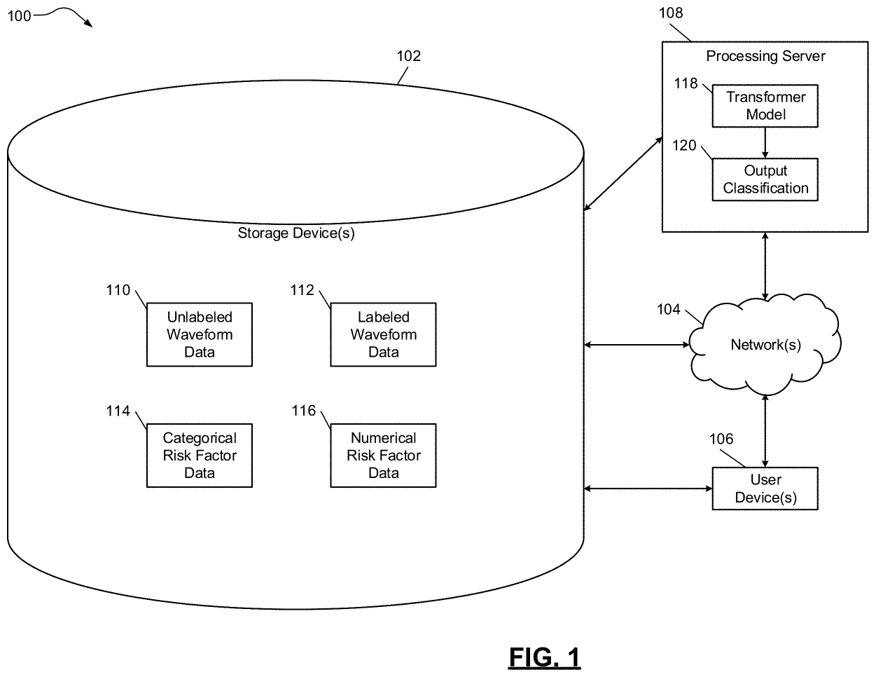

FIG. 1 is a block diagram of an example implementation of a system 100 for analyzing and detecting waveforms using a machine learning transformer model, including a storage device 102. While the storage device 102 is generally described as being deployed in a computer network system, the storage device 102 and/or components of the storage device 102 may otherwise be deployed (for example, as a standalone computer setup, etc.). The storage device 102 may be part of or include a desktop computer, a laptop computer, a tablet, a smartphone, a HDD device, a SDD device, a RAID system, a SNA system, a NAS system, a cloud device, etc.

As shown in FIG. 1, the storage device 102 includes unlabeled waveform data 110, labeled waveform data 112, categorical risk factor data 114, and numerical risk factor data 116. The unlabeled waveform data 110, labeled waveform data 112, categorical risk factor data 114, and numerical risk factor data 116 may be located in different physical memories within the storage device 102, such as different random access memory (RAM), read-only memory (ROM), a non-volatile hard disk or flash memory, etc. In some implementations, one or more of the unlabeled waveform data 110, labeled waveform data 112, categorical risk factor data 114, and numerical risk factor data 116 may be located in the same memory (e.g., in different address ranges of the same memory, etc.).

As shown in FIG. 1, the system 100 also includes a processing server 108. The processing server 108 may access the storage device 102 directly, or may access the storage device 102 through one or more networks 104. Similarly, a user device 106 may access the processing server 108 directly or through the one or more networks 104.

The processing server includes a transformer model 118, which produces an output classification 120. A local device including the storage device 102 may send raw waveform data, or compress the waveform data through FFT, DCT or another compression technique and send the compressed data, along with additional risk factors, daily activity, etc., to the processing server 108. The transformer model 118 may receive the unlabeled waveform data 110, labeled waveform data 112, categorical risk factor data 114, and numerical risk factor data 116, and output an output classification 120. As described further below, the transformer model 118 may include a BERT model, an encoder-decoder model, etc.

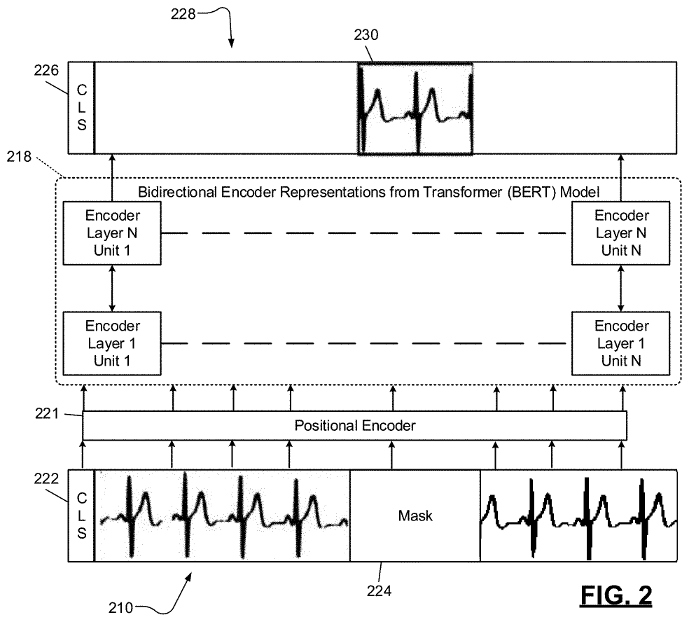

The unlabeled waveform data 110 may include general waveforms that can be used to pre-train the transformer model 118. The unlabeled waveform data 110 (e.g., unlabeled waveform training data) may not include specific classifications, identified waveform characteristics, etc., and may be used to generally train the transformer model 118 to handle the type of waveforms that are desired for analysis. As described further below and with reference to FIG. 2, the unlabeled waveform data 110 may be supplied as an input to the transformer model 118 with randomly applied input masks, where the transformer model 118 is trained to predict the masked portion of the input waveform.

The unlabeled waveform data 110 may be particularly useful when there is a much larger amount of general waveform data as compared to a smaller amount of specifically classified labeled waveform data 112 (e.g., labeled waveform training data). For example, an abundant amount of general ECG waveforms (e.g., the unlabeled waveform data 110) may be obtained by downloading from websites such as PhysioNet, ECG View, etc., while a ECGs that are specifically classified with labels (e.g., the labeled waveform data 112) such as heart arrhythmias, P and T waves, etc., may be much smaller. Pre-training the transformer model 118 with the larger amount of unlabeled waveform data 110 may improve the accuracy of the transformer model 118, which can then be fine-tuned by training with the smaller amount of labeled waveform data 112. In other words, the transformer model 118 may be pre-trained to accurately predict ECG waveforms in general, and then fine-tuned to classify a specific ECG feature such as a heart arrhythmia, P and T waves, etc.

As shown in FIG. 1, the storage device 102 also includes categorical risk factor data 114 and numerical risk factor data 116. The categorical risk factor data 114 and the numerical risk factor data 116 may be used in addition to the unlabeled waveform data 110 and the labeled waveform data 112, to improve the diagnostic accuracy of the output classification 120 of the transformer model 118. For example, in addition to ECG waveforms, many sensor signals such as patient vital signs, patient daily activity, patient risk factors, etc., may help improve the diagnostic accuracy the diagnostic accuracy of the output classification 120 of the transformer model 118. Categorical risk factor data 114 may include a sex of the patient, etc., while the numerical risk factor data 116 may include a patient age, weight, height, etc.

A system administrator may interact with the storage device 102 and the processing server 108 to implement the waveform analysis via a user device 106. The user device 106 may include a user interface (UI), such as a web page, an application programming interface (API), a representational state transfer (RESTful) API, etc., for receiving input from a user. For example, the user device 106 may receive a selection of unlabeled waveform data 110, labeled waveform data 112, categorical risk factor data 114, and numerical risk factor data 116, a type of transformer model 118 to be used, a desired output classification 120, etc. The user device 106 may include any suitable device for receiving input and classification outputs 120 to a user, such as a desktop computer, a laptop computer, a tablet, a smartphone, etc. The user device 106 may access the storage device 102 directly, or may access the storage device 102 through one or more networks 104. Example networks may include a wireless network, a local area network (LAN), the Internet, a cellular network, etc.

### Training the Transformer Model

FIG. 2 illustrates an example transformer model 218 for use in the system 100 of FIG. 1. As shown in FIG. 2, the transformer model 218 is a Bidirectional Encoder Representations from Transformers (BERT) model. One example BERT model is described in “BERT: Pre-training of Deep Bidirectional Transformers for Language Understanding” by Devlin et al., (24 May 2019) at https://arxiv.org/abs/1810.04805. For example, the BERT model may include multiple encoder layers or blocks, each having a number or elements. The model 218 may also include feed-forward networks and attention heads connected with the encoder layers, and back propagation between the encoder layers. While the BERT model was developed for use in language processing, example techniques described here use the BERT model in non-traditional ways that are departures from normal BERT model use, e.g., by analyzing patient sensor waveform data such as ECGs, etc.

As shown in FIG. 2, the unlabeled waveform data 210 is supplied to the input of the transformer model 218 to pre-train the model 218. For example, the unlabeled waveform data 210 may include general ECG waveforms used to train the model to accurately predict ECG waveform features. The unlabeled waveform data 210 includes a special input token 222 (e.g., [CLS] which stands for classification). The unlabeled waveform data 210 also includes a mask 224.

The unlabeled waveform data 210 may include electrical signals from N electrodes at a given time t, which forms a feature vector with size N. For example the input may include voltage readings from up to twelve leads of an ECG recording. An example input vector of size 3 is shown below in Equation 1, for three time steps:

$$[ \begin{matrix}0.1 mV & 0.11 mV & 0.12 mV \\ 0.09 mV & 0.1 mV & 0.11 mV \\ 0.4 mV & 0.6 mV & 0.7 mV\end{matrix} ] \qquad ( Equation 1 )$$

The waveform may have any suitable duration, such as about ten beats, several hundred beats, etc. A positional encoder 221 applies time stamps to the entire time series to maintain the timing relationship of the waveform sequence. In various implementations, a fully connected neural network (e.g., adapter) converts the positional encoded vector to a fixed-size vector. The size of vector is determined by model dimension. In some implementations, an FFT compression block may compress the waveform data 210 and supply the FFT compression directly to the transformer model 218. In that case, the FFT compression may be placed in different time range bins of the waveform data 210, for supplying to different input blocks of the transformer model 218.

The masks 224 are applied at randomly selected time intervals time intervals [t1+Δt, t2+Δt, . . . ]. The modified input is then fed into the BERT model 218. The model 218 is trained to predict the output signal portions 230 corresponding to the masked intervals [t1+Δt, t2+Δt, . . . ], in the output 228. For example, the transformer model 218 may take the input and flow the input through a stack of encoder layers. Each layer may apply self-attention, and then pass its results through a feed-forward network before handing off to the next encoder layer. Each position in the model outputs a vector of a specified size. In various implementations, the focus is on the output of the first position where the CLS token 222 was passed (e.g., a focus on the CLS token 226 in the output 228). The output CLS token 226 may be for a desired classifier. For example, the CLS token 226 may be fed through a feed-forward neural network and a softmax to provide a class label output.

Although the output 228 includes an output token 226 (e.g., a CLS token) in the pre-training process, the primary goal of pre-training the model 218 with the unlabeled waveform data 210 may be to predict the output signal portions 230 to increase the accuracy of the model 218 for processing ECG signals. Because no label is required for the ECG data during pre-training, the pre-trained model 218 may be agnostic to an underlying arrhythmia, condition, disease, etc.

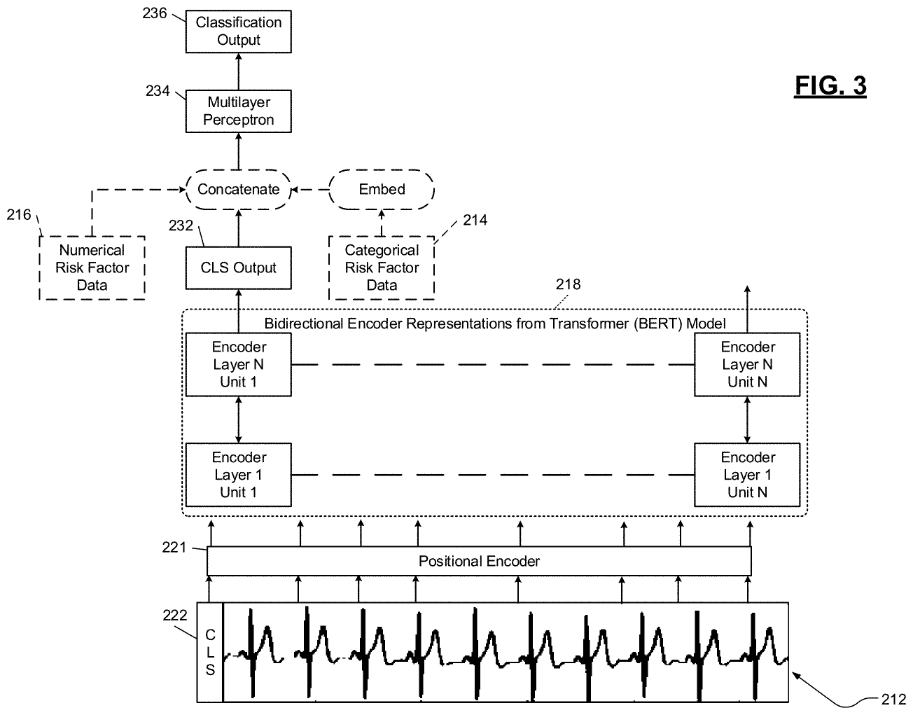

FIG. 3 illustrates a process of fine-tuning the transformer model 218 using labeled waveform data 212. For example, the labeled waveform data 212 may include ECG waveforms that have been identified as having heart arrhythmias, ECG waveforms with identified P and T waves, etc. The labeled waveform data 212 is supplied to the transformer model 218 without using any masks.

The CLS output 232 feeds into a multilayer fully connected neural network, such as a multilayer perceptron (MLP) 234. A softmax function for a categorical label is applied, or an Li distance for a numerical label is applied, to generate a classification output 236. An example softmax function is shown below in Equations 2 and 3:

$$\begin{matrix}L = - {\sum }_{i} {y}_{i} log ( {p}_{i} ) & ( Equation 2 ) \\ {p}_{i} = \frac{{e}^{{a}_{i}}}{{\sum }_{k = 1}^{N} {e}^{{a}_{k}}} & ( Equation 3 )\end{matrix}$$

where yᵢ is a label, Equation 3 is a softmax probability, and aᵢ is the logit output from the MLP 234. Because the transformer model 218 has already been pre-trained with unlabeled waveform data 210, the dataset for the labeled waveform data 212 may be smaller while still adequately fine-tuning the model.

In various implementations, categorical risk factor data 214 and numerical risk factor data 216 may be integrated with the waveform analysis of the transformer model 218. As shown in FIG. 3, optionally the categorical risk factor data 214 is first embedded into a vector representation. For example, integers representing different category values may be converted to a one-hot encoding representation and fed into a one or multiple layer fully connected neural network. The output is a fixed size feature vector. This procedure is called categorical feature embedding. An example vector for male or female patients and smoker or non-smoker patients is illustrated below in Table 1.

TABLE 1

| | Female | Male | Smoker | Non-smoker |
|---|---|---|---|---|
| Smoker (M) | 0 | 1 | 1 | 0 |
| Non-Smoker (F) | 1 | 0 | 0 | 1 |

The embedded vector may be concatenated with the numerical risk factor data 216 and the CLS output 232. The concatenated vector including the embedded categorical risk factor data, the numerical risk factor data 216 and the CLS output 232, is then supplied to the MLP 234. Therefore, the numerical risk factor data 216 and the categorical risk factor data 214 may enhance the classification output 236.

Although FIG. 3 illustrates concatenating the numerical risk factor data 216 and the categorical risk factor data 214 with the CLS output 232 prior to the MLP 234, in various implementations the numerical risk factor data 216 and the categorical risk factor data 214 may be incorporated at other locations relative to the transformer model 218. For example, after embedding the categorical risk factor data 216, the embedded vector may be concatenated with the numerical risk factor data 216 and the labeled waveform data 212 prior to supplying the data as an input to the transformer model. The concatenated vector may be encoded with time stamps for positional encoding via a positional encoder 221, and then supplied as input to the transformer model 218.

When the CLS output 232 has a categorical value, the loss function may use a softmax function L, such as the function shown below in Equations 4 and 5:

$$\begin{matrix}L = - {\sum }_{i} {y}_{i} log ( {p}_{i} ) & ( Equation 4 ) \\ {p}_{i} = \frac{{e}^{{a}_{i}}}{{\sum }_{k = 1}^{N} {e}^{{a}_{k}}} & ( Equation 5 )\end{matrix}$$

where yᵢ is a label, Equation 3 is a softmax probability, and aᵢ is the logit output from the MLP 234.

Although FIGS. 2 and 3 illustrate a BERT model that is pre-trained with unlabeled waveform data 210 and then fine-tuned with labeled waveform data 212, in various implementations there may be enough labeled waveform data that pre-training with the unlabeled waveform data 210 is unnecessary. Also, in various implementations, other transformer models may be used, such as encoder-decoder transformers, etc.

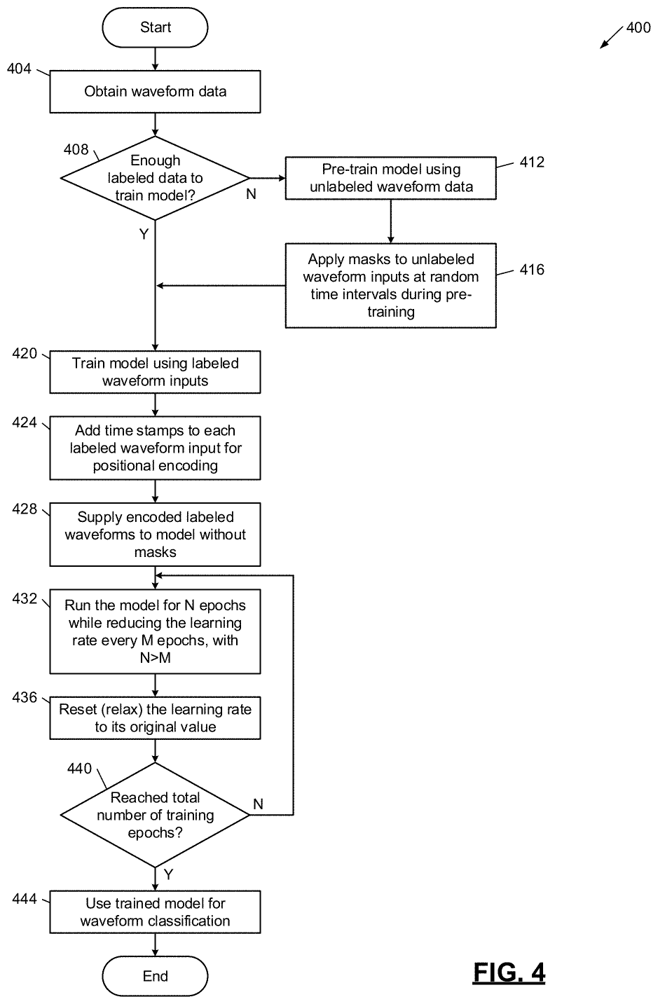

FIG. 4 is a flowchart depicting an example method 400 of training a waveform analysis transformer model. Although the example method is described below with respect to the system 100, the method may be implemented in other devices and/or systems. At 404, control begins by obtaining waveform data for analysis. The control may be any suitable processor, controller, etc.

At 408, control determines whether there is enough labeled data to train the transformer model. There are often much larger data sets available for unlabeled, general waveforms in the area of interest, as compared to labeled waveforms that have identified specific properties about the waveform. For example, there may be hundreds of millions of general ECG waveforms available for download, but a much smaller amount of ECG waveforms that have been labeled with specific identifiers such as a heart arrhythmia, P and T waves, etc.

If there is not sufficient labeled data at 408, control proceeds to 412 to pre-train the model using the unlabeled waveform data at 412. Specifically, at 416, control applies masks to the unlabeled waveform inputs at random time intervals during the pre-training, and the transformer model trains its ability to accurately predict the masked portions of the waveform.

Control then proceeds to 420 to train the model using the labeled waveform inputs (e.g., to fine-tune the model using the labeled waveform inputs). If there is already sufficient labeled waveform data at 408 to train the model, control can proceed directly to 420 and skip the pre-training steps 412 and 416. At 424, control adds time stamps to each labeled waveform for position encoding. The encoded labeled waveforms are then supplied to the model without masks at 428.

Next, the transformer model is run for N epochs while reducing the learning rate every M epochs, with N>M, at 432. The learning rate is then reset (e.g., relaxed) to its original value at 436. For example, an Adam optimizer may be used with an initial learning rate of 0.0001, where rest hyper-parameters are the same between different epochs. Each training could have 200 epochs, where a scheduler steps down the learning rate by 0.25 for every 50 epochs. After the 200 epochs are completed, the learning rate may be reset (e.g., relaxed) back to 0.0001.

At 440, control determines whether the total number of training epochs has been reached. If not, control returns to 432 to run the model for N epochs again, starting with the reset learning rate. Once the total number of training epochs has been reached at 440, control proceeds to 444 to use the trained model for analyzing waveforms.

As described above, instead of using a continuously reduced learning rate throughout training, the learning rate may be relaxed periodically to improve training of the transformer model. For example, the training process may include five relaxations, ten relaxations, forty relaxations, etc. The amount of relaxations in the training process may be selected to avoid overtraining the model, depending on the amount of data available for training. Training accuracy may continue to improve as the number of relaxations increases, although testing accuracy may stop improving after a fixed number of relaxations, which indicates that the transformer model may be capable of overfitting. The relaxation adjustment may be considered as combining pre-training and fine-tuning of the model, particularly where there is not enough data for pre-training. In various implementations, the transformer model may use periodic relaxation of the learning during pre-training with unlabeled waveform data, during fine-tuning training with labeled waveform data, etc.

### Analyzing ECG Waveforms

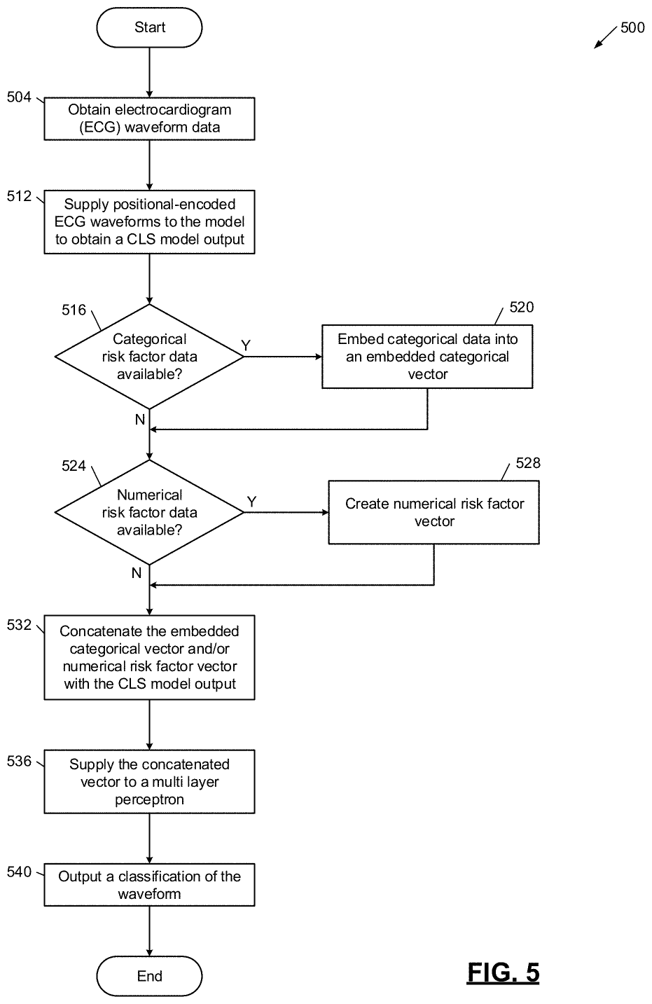

FIG. 5 is a flowchart depicting an example method 500 of using a transformer model to analyze ECG waveforms. Although the example method is described below with respect to the system 100, the method may be implemented in other computing devices and/or systems. At 504, control begins by obtaining ECG waveform data (e.g., ECG waveform data from a scan of a specific patient, etc.), which may be considered as a target waveform. The ECG waveform data may be stored in files of voltage recordings from one or more sensors over time, in a healthcare provider database, publicly accessible server with de-identified example waveforms, etc. Control adds time stamps to the ECG waveform inputs for position encoding, and the positional-encoded ECG waveform input is supplied to the model at 512 to obtain a CLS model output.

At 516, control determines whether categorical risk factor data is available. For example, whether the sex of the patient is known, etc. If so, the categorical risk factor data is embedded into an embedded categorical vector at 520. An example of categorical risk factor data is shown above in Table 1.

Control then proceeds to 524 to determine whether numerical risk factor data is available. Example numerical risk factor data may include an age of the patient, a height of the patient, a weight of the patient, etc. If so, control creates a numerical risk factor vector at 528.

At 532, control concatenates the embedded categorical risk factor vector and/or the numerical risk factor vector with the CLS model output. The concatenated vector is then supplied to a multilayer perceptron (MLP) at 536, and control outputs a classification of the waveform at 540. For example, the output classification may be an indication of whether a heart arrhythmia exists, a diagnosis of a condition of the patient, a location of P and T waves in the waveform, etc.

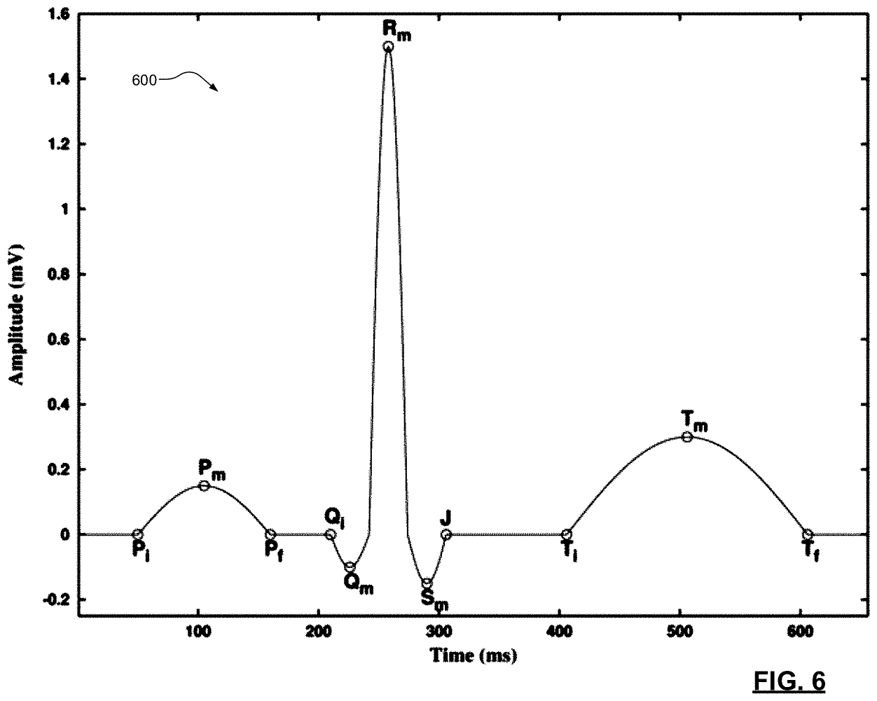

FIG. 6 illustrates an example ECG waveform 600 depicting P and T waves. The R wave may be detected reliably using a Pan Tompkins algorithm, etc. However, P and T wave detection is difficult due to the noise, smaller and wider shapes of the P and T waves, etc.

In various implementations, the Pan Tompkins algorithm may be used to detect the R wave and then to generate a data sequence for the waveform (e.g., centered around the detected R wave, using the detected R wave as a base reference point, etc.).

The generated data sequence of the ECG waveform is then fed to a transformer to fine-tune a model for detecting P and T waves. For example, the transformer model may first be pre-trained with general ECG waveforms. Then, a cardiologist labels fiducial points (e.g., eleven fiducial points, etc.) on each ECG waveform when supplying the labeled waveform data to fine-tune the model.

In various implementations, the input to the transformer encoder is the ECG data, and the output is the fiducial points (e.g., eleven fiducial points, more or less points, etc.). A typical cycle of an ECG with normal sinus rhythm is shown in FIG. 6, with P, Q, R, S and T waves. In this example, the starting and ending points of the P and T waves are labeled as Pᵢ, P_{f}, Tᵢ, and T_{f}, and the maximums of each wave are labeled as Pₘ and Tₘ, respectively, as described by Yáñez de la Rivera et al., “Electrocardiogram Fiducial Points Detection and Estimation Methodology for Automatic Diagnose,” The Open Bioinformatics Journal Vol. 11, pp. 208-230 (2018). The starting point of the QRS complex is labeled Qᵢ, and the ending point is labeled as J. The maximum/minimum of the Q, R and S waves are labeled as Qₘ, Rₘ and Sₘ, respectively.

The portion of the signal between two consecutive Rₘ points is known as the RR interval. Furthermore, the portion of the signal between Pᵢ and the following Qᵢ point is known as the PQ (or PR) interval, and the portion of the signal between Qᵢ and the following T_{f} point is known as the QT interval. Analogously, the portion of the signal between the J point and the following Tᵢ point is known as the ST segment, and the portion of the signal between P_{f} and the following Qᵢ point is known as the PQ segment. In various implementations, the output classification of the transformer model may include fiducial points of the input ECG waveform, which may be used to identify P and T waves.

Because fiducial points are continuous variables over time, a loss function L may be defined as shown in Equation 6:

$$L = {\sum }_{i = 0}^{i = 10} {‖ {t}_{i} - {t}_{i}^{g} ‖}_{1} \qquad ( Equation 6 )$$

where tᵢ^{g} is on fiducial point label (e.g., ground truth), while tᵢ is an output block of the transformer model. An example output that includes eleven fiducial points is illustrated below in Equation 7, with timestamps for each of the eleven points.

[0.04 s 0.06 s 0.1 s 0.11 s 0.13 s 0.16 s 0.21 s 0.25 s 0.3 s 0.34 s 0.36 s] (Equation 7)

### Additional Use Cases

In various implementations, the transformer models described herein may be used to analyze a variety of different types of waveforms, in a wide range of frequencies from low frequency sound waves or seismic waves to high frequency optical signals, etc. The signal could be aperiodic, as long as a pattern exists in the data and a sensor device is able to capture the signal with sufficient resolution.

In various implementations, a transformer model may be used to analyze seismograph waveforms for earthquake detection. Although seismograph stations monitor for earthquakes continuously, earthquake events are rare. In order to address this unbalanced classes issue, the transformer model may first be pre-trained with daily seismograph waveforms. The daily seismograph waveforms may be unlabeled (e.g., not associated with either an earthquake event or no earthquake event). A portion of the daily seismograph waveforms may be masked, so that the model first learns to predict normal seismograph waveform features.

Next, available earthquake event data may be used to fine-tune the detector. For example, seismograph waveforms that have been classified as either an earthquake event or no earthquake event may be supplied to train the model to predict earthquake events. Once the model has been trained, live seismograph waveforms may be supplied to the model to predict whether future earthquake events are about to occur. Additional geophysical information can also be integrated into the transformer model to create categorization vectors, such as aftershock occurrences, distances from known fault lines, type of geological rock formations in the area, etc.

A transformer model may be used to analyze automobile and human traffic pattern waveforms. This input waveforms of automobile and human traffic may be combined with categorical data such as weekdays, holidays, etc., may be combined with numerical data such as weather forecast information, etc. The transformer model may be used to output a pattern classification of the automobile and human traffic.

For example, the model may be pre-trained with a waveform including a number of vehicles or pedestrians over time, using masks, to train the model to predict traffic waveforms. The model may then be fine-tuned with waveforms that have been classified as high traffic, medium traffic, low traffic, etc., in order to predict future traffic patterns based on live waveforms of vehicle or pedestrian numbers. In various implementations, waveforms of vehicle or pedestrian numbers in one location may be used to predict a future traffic level in another location.

In various implementations, the transformer model may be used for analyzing medical waveform measurements, such as an electroencephalogram (EEG) waveform based on readings from multiple sensors, to assist in control for manipulating artificial limbs, to provide diagnostics for depression and Alzheimer's disease, etc.

For example, similar to the ECG cases described herein, a model may be pre-trained with unlabeled EEG data to first train the model to predict EEG waveforms using masks. The model may then be fine-tuned with EEG waveforms that have been classified as associated with depression, Alzheimer's disease, etc., in order to predict certain conditions from the EEG waveforms.

The transformer model may be used for network data flow analysis. For example, waveforms of data traffic in a network may be supplied to a transformer model in order to detect recognized patterns in the network, such as anomalies, dedicated workflows, provisioned use, etc. Similar to other examples, unlabeled waveforms of network data flows may first be provided to pre-train the model to predict network data flow waveforms over time using masks, and then labeled waveform data may be used to fine-tune the model by supplying network data flow waveforms that have been classified as an anomaly, as a dedicated workflow, as a provisioned use, etc.

In various implementations, the transformer model may be used for analysis of waveforms having small frequencies and long wavelengths. For example, the transformer model may receive solar activity waveforms as inputs, and classify recognized patterns of solar activity as an output. As another example, weather waveforms could be supplied as inputs to the model in order to output classifications of recognized weather patterns. For example, the model may be trained to classify a predicted next day weather pattern as cloudy, partly cloudy, sunny, etc.

The transformer model may be used to predict current subscribers (for example, to a newspaper, to a streaming service, or to a periodic delivery service), that are likely to drop their subscriptions in the next period, such as during the next month or the next year. This may be referred to as a subscriber churn. The model prediction may be used by a marketing department to focus on the highest likelihood of churning subscribers for most effective targeting of their subscriber retention efforts.

For example, if an average of 5,000 subscribers churn each month out of a total of 500,000 subscribers, randomly selecting 1,000 subscribers for retention efforts would typically result in only reaching 10 subscribers that were going to churn. However, if a model has, for example, 40% prediction accuracy, there would be an average of 400 subscribers planning to churn in the group of 1,000, which is a much better cohort for the marketing term to focus on.

Inputs to the model may be obtained from one or more data sources, which may be linked by an account identifier. In various implementations, input variables may have a category type, a numerical type, or a target type. For example, category types may include a business unit, a subscription status, an automatic renewal status, a print service type, an active status, a term length, or other suitable subscription related categories. Numerical types may include a subscription rate (which may be per period, such as weekly). Target types may include variables such as whether a subscription is active, or other status values for a subscription.

In various implementations, a cutoff date may be used to separate training and testing data, such as a cutoff date for subscription starts or weekly payment dates. Churners may be labeled, for example, where a subscription expiration date is prior to the cutoff date and a subscription status is false, or where an active value is set to inactive.

For each labeled churner, input data may be obtained by creating a payment end date that is a specified number of payments prior to the expired date (such as dropping the last four payments), and setting a payment start date as a randomly selected date between, for example, one month and one year prior to the payment end. For each labeled subscriber, the payment end date may be set, for example, one month prior to the cutoff date to avoid bias. The payment start date may be selected randomly between, for example, one month and one year from the payment end date.

Two datasets may be generated using cutoff dates that are separated from one another by, for example one month. Training and evaluation datasets are built using the two different cutoff dates. All accounts that are subscribers at the first cutoff date may be selected when the account payment end date is close to the first cutoff date and the target label indicates the subscription is active. Next, target labels may be obtained for subscribers at the first cutoff date that are in the second cutoff date dataset.

For example, all subscriber target labels in the first cutoff date dataset may indicate active subscriptions, while some of the target labels in the second cutoff date dataset will indicate churners. Testing dataset target labels may then be replaced with labels generated by finding the subscribers at the first cutoff date that are in the second cutoff date dataset.

In various implementations, a transformer model data complex may be built by converting categorical data to a one-dimensional vector with an embedding matrix, and normalizing each one-dimensional vector. All one-dimensional vectors are concatenated, and the one-dimensional vector size is fixed to the model size.

The transformer encoder output and attribute complex output sizes may be, for example, (B, 256), where B is a batch size. The payment sequence may contain a list of payment complex (N, B, 256), where N is a number of payments in the sequence. In various implementations, multi-layer perception of the model may include an input value of 512, an output value of 2, and two layers (512, 260) and (260, 2). A transformer encoder may be implemented using a classifier of ones (B, 256), a separator of zeros (B, 256), PCn inputs of a Payment Complex n (B, 256), and a classifier output of (B, 256). The model dimension may be 256, with a forward dimension value of 1024 and a multi-head value of 8.

### Computer Device

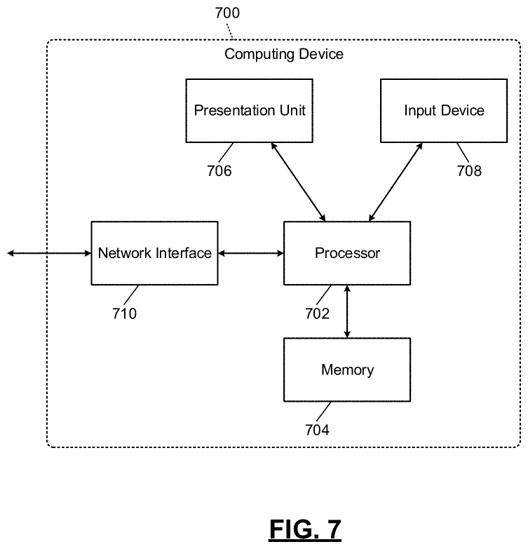

FIG. 7 illustrates an example computing device 700 that can be used in the system 100. The computing device 700 may include, for example, one or more servers, workstations, personal computers, laptops, tablets, smartphones, gaming consoles, etc. In addition, the computing device 700 may include a single computing device, or it may include multiple computing devices located in close proximity or distributed over a geographic region, so long as the computing devices are specifically configured to operate as described herein. In the example implementation of FIG. 1, the storage device(s) 102, network(s) 104, user device(s) 106, and processing server(s) 108 may each include one or more computing devices consistent with computing device 700. The storage device(s) 102, network(s) 104, user device(s) 106, and processing server(s) 108 may also each be understood to be consistent with the computing device 700 and/or implemented in a computing device consistent with computing device 700 (or a part thereof, such as, e.g., memory 704, etc.). However, the system 100 should not be considered to be limited to the computing device 700, as described below, as different computing devices and/or arrangements of computing devices may be used. In addition, different components and/or arrangements of components may be used in other computing devices.

As shown in FIG. 7, the example computing device 700 includes a processor 702 including processor hardware and a memory 704 including memory hardware. The memory 704 is coupled to (and in communication with) the processor 702. The processor 702 may execute instructions stored in memory 704. For example, the transformer model may be implemented in a suitable coding language such as Python, C/C++, etc., and may be run on any suitable device such as a GPU server, etc.

A presentation unit 706 may output information (e.g., interactive interfaces, etc.), visually to a user of the computing device 700. Various interfaces (e.g., as defined by software applications, screens, screen models, GUIs etc.) may be displayed at computing device 700, and in particular at presentation unit 706, to display certain information to the user. The presentation unit 706 may include, without limitation, a liquid crystal display (LCD), a light-emitting diode (LED) display, an organic LED (OLED) display, an “electronic ink” display, speakers, etc. In some implementations, presentation unit 706 may include multiple devices. Additionally or alternatively, the presentation unit 706 may include printing capability, enabling the computing device 700 to print text, images, and the like on paper and/or other similar media.

In addition, the computing device 700 includes an input device 708 that receives inputs from the user (i.e., user inputs). The input device 708 may include a single input device or multiple input devices. The input device 708 is coupled to (and is in communication with) the processor 702 and may include, for example, one or more of a keyboard, a pointing device, a mouse, a stylus, a touch sensitive panel (e.g., a touch pad or a touch screen, etc.), or other suitable user input devices. In various implementations, the input device 708 may be integrated and/or included with the presentation unit 706 (for example, in a touchscreen display, etc.). A network interface 710 coupled to (and in communication with) the processor 702 and the memory 704 supports wired and/or wireless communication (e.g., among two or more of the parts illustrated in FIG. 1).

### CONCLUSION

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the implementations is described above as having certain features, any one or more of those features described with respect to any implementation of the disclosure can be implemented in and/or combined with features of any of the other implementations, even if that combination is not explicitly described. In other words, the described implementations are not mutually exclusive, and permutations of one or more implementations with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. The phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WWI wireless networking standard) and IEEE Standard 802.3-2015 (also known as the ETHERNET wired networking standard). Examples of a WPAN are IEEE Standard 802.15.4 (including the ZIGBEE standard from the ZigBee Alliance) and, from the Bluetooth Special Interest Group (SIG), the BLUETOOTH wireless networking standard (including Core Specification versions 3.0, 4.0, 4.1, 4.2, 5.0, and 5.1 from the Bluetooth SIG).

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A computerized method of analyzing a waveform using a machine learning transformer model, the method comprising:
obtaining labeled waveform training data and unlabeled waveform training data;
supplying the unlabeled waveform training data to a transformer model to pre-train the transformer model by masking a portion of an input to the transformer model;
supplying the labeled waveform training data to the transformer model without masking a portion of the input to the transformer model to fine-tune the transformer model, wherein each waveform in the labeled waveform training data includes at least one label identifying a feature of the waveform; and
supplying a target waveform to the transformer model to classify at least one feature of the target waveform, wherein the at least one classified feature corresponds to the least one label of the labeled waveform training data.

2. The method of claim 1, further comprising:
obtaining categorical risk factor data;
obtaining numerical risk factor data;
embedding categorical risk factor data and concatenating the embedded categorical risk factor data with the numerical risk factor data to form a concatenated feature vector; and
supplying the concatenated feature vector to the transformer model to increase an accuracy of the at least one classified feature.

3. The method of claim 2, wherein:
the unlabeled waveform training data, the labeled waveform training data, and the target waveform each comprise an electrocardiogram (ECG) waveform recorded from a patient;
the categorical risk factor data includes a sex of the patient; and
the numerical risk factor data includes at least one of an age of the patient, a height of the patient, and a weight of the patient.

4. The method of claim 2, wherein:
the categorical risk factor data includes multiple groups of categorical values;
each group is encoded using one-hot encoding; and
embedding the categorical risk factor data includes combining each of the encoded groups into a combined encoded vector and then feeding the combined encoded vector to a neural network to output an embedded categorical risk factor vector.

5. The method of claim 1, wherein:
the unlabeled waveform training data, the labeled waveform training data, and the target waveform each comprise an electrocardiogram (ECG) waveform recorded from a patient;
the at least one label of each waveform in the labeled waveform training data includes at least one of a detected heart arrhythmia, a P wave and a T wave; and
the at least one classified feature includes the at least one of a detected heart arrhythmia, a P wave and a T wave.

6. The method of claim 1, wherein the transformer model comprises a Bidirectional Encoder Representations from Transformers (BERT) model.

7. The method of claim 1, wherein supplying the unlabeled waveform training data to pre-train the transformer model and supplying the labeled waveform training data to fine-tune the transformer model each include periodically relaxing a learning rate of the transformer model by reducing the learning rate during a specified number of epochs and then resetting the learning rate to an original value before running a next specified number of epochs.

8. The method of claim 1, wherein:
the unlabeled waveform training data includes daily seismograph waveforms;
the labeled waveform training data includes detected earthquake event seismograph waveforms; and
the at least one classified feature includes a detected earthquake event.

9. The method of claim 1, wherein the labeled waveform training data, the unlabeled waveform training data, and the target waveform each include at least one of an automobile traffic pattern waveform, a human traffic pattern waveform, an electroencephalogram (EEG) waveform, a network data flow waveform, a solar activity waveform, and a weather waveform.

10. The method of claim 1, wherein:
the transformer model is located on a processing server;
the target waveform is stored on a local device separate from the processing server; and
the method further includes compressing the target waveform and transmitting the target waveform to the processing server for input to the transformer model.

11. A computer system comprising:
memory hardware configured to store unlabeled waveform training data, labeled waveform training data, a target waveform, a transformer model, and computer-executable instructions; and
processor hardware configured to execute the instructions, wherein the instructions include:
obtaining labeled waveform training data and unlabeled waveform training data;
supplying the unlabeled waveform training data to the transformer model to pre-train the transformer model by masking a portion of an input to the transformer model;
supplying the labeled waveform training data to the transformer model without masking a portion of the input to the transformer model to fine-tune the transformer model, each waveform in the labeled waveform training data including at least one label identifying a feature of the waveform; and
supplying a target waveform to the transformer model to classify at least one feature of the target waveform, wherein the at least one classified feature corresponds to the least one label of the labeled waveform training data.

12. The computer system of claim 11, wherein the instructions include:
obtaining categorical risk factor data;
obtaining numerical risk factor data;
embedding categorical risk factor data and concatenating the embedded categorical risk factor data with the numerical risk factor data to form a concatenated feature vector; and
supplying the concatenated feature vector to the transformer model to increase an accuracy of the at least one classified feature.

13. The computer system of claim 12, wherein:
the unlabeled waveform training data, the labeled waveform training data, and the target waveform each comprise an electrocardiogram (ECG) waveform recorded from a patient;
the categorical risk factor data includes a sex of the patient; and
the numerical risk factor data includes at least one of an age of the patient, a height of the patient, and a weight of the patient.

14. The computer system of claim 12, wherein:
the categorical risk factor data includes multiple groups of categorical values;
each group is encoded using one-hot encoding; and
embedding the categorical risk factor data includes combining each of the encoded groups into a combined encoded vector and then feeding the combined encoded vector to a neural network to output an embedded categorical risk factor vector.

15. The computer system of claim 11, wherein:
the unlabeled waveform training data, the labeled waveform training data, and the target waveform each comprise an electrocardiogram (ECG) waveform recorded from a patient;
the at least one label of each waveform in the labeled waveform training data includes at least one of a detected heart arrhythmia, a P wave and a T wave; and
the at least one classified feature includes the at least one of a detected heart arrhythmia, a P wave and a T wave.

16. A non-transitory computer-readable medium storing processor-executable instructions, the instructions comprising:
obtaining labeled waveform training data and unlabeled waveform training data;
supplying the unlabeled waveform training data to a transformer model to pre-train the transformer model by masking a portion of an input to the transformer model;
supplying the labeled waveform training data to the transformer model without masking a portion of the input to the transformer model to fine-tune the transformer model, each waveform in the labeled waveform training data including at least one label identifying a feature of the waveform; and
supplying a target waveform to the transformer model to classify at least one feature of the target waveform, wherein the at least one classified feature corresponds to the least one label of the labeled waveform training data.

17. The non-transitory computer-readable medium of claim 16, wherein supplying the unlabeled waveform training data to pre-train the transformer model and supplying the labeled waveform training data to fine-tune the transformer model each include periodically relaxing a learning rate of the transformer model by reducing the learning rate during a specified number of epochs and then resetting the learning rate to an original value before running a next specified number of epochs.

18. The non-transitory computer-readable medium of claim 16, wherein:
the unlabeled waveform training data includes daily seismograph waveforms;
the labeled waveform training data includes detected earthquake event seismograph waveforms; and
the at least one classified feature includes a detected earthquake event.

19. The non-transitory computer-readable medium of claim 16, wherein the labeled waveform training data, the unlabeled waveform training data, and the target waveform each include at least one of an automobile traffic pattern waveform, a human traffic pattern waveform, an electroencephalogram (EEG) waveform, a network data flow waveform, a solar activity waveform, and a weather waveform.

20. The non-transitory computer-readable medium of claim 16, wherein:
the transformer model is located on a processing server;
the target waveform is stored on a local device separate from the processing server; and
the instructions further include compressing the target waveform and transmitting the target waveform to the processing server for input to the transformer model.
